(19) United States
(12) Patent Application Publication
(10) Pub. No.: US 2025/0295656 A1
(43) Pub. Date: Sep. 25, 2025

(54) COMBINATION OF LURBINECTEDIN AND IMMUNE CHECKPOINT INHIBITORS

(71) Applicant: PHARMA MAR, S.A., Madrid (ES)

(72) Inventors: Guido Kroemer, Villejuif (FR); Oliver Kepp, Villejuif (FR)

(21) Appl. No.: 19/190,098

(22) Filed: Apr. 25, 2025

Related U.S. Application Data
(63) Continuation of application No. 18/023,804, filed on Feb. 28, 2023.

(30) Foreign Application Priority Data
Sep. 4, 2020 (WO) PCT/EP2020/074860

(51) Int. Cl. A61K 31/4995; A61K 39/00; A61K 39/395; A61P 35/00

(52) U.S. Cl. CPC A61K 31/4995; A61K 39/3955; A61P 35/00; A61K 2039/505; A61K 2039/507

(57) ABSTRACT

Described are combination therapies for the treatment of solid tumours, said therapies comprising lurbinectedin and an immune checkpoint inhibitor, for example, anti-PD-1, anti-PD-L1 or anti-CTLA-4 antibodies.

### CROSS REFERENCE TO RELATED APPLICATIONS

This application is a continuation of U.S. application Ser. No. 18/023,804, filed on Feb. 28, 2023, which is a U.S. National Stage Application filed under 35 U.S.C. § 371, based on International Patent Application No. PCT/EP 2021/074425, filed on Sep. 3, 2021, which claims priority to International Patent Application No. PCT/EP 2020/074860, filed on Sep. 4, 2020. The entire contents of each of the above applications are incorporated herein by reference.

### FIELD OF THE INVENTION

The present invention relates to therapeutic treatment of cancers, particularly solid tumours, with combination therapy using lurbinectedin and immune checkpoint inhibitors.

### BACKGROUND TO THE INVENTION

Immune checkpoint inhibitor (ICI) therapy is a form of cancer immunotherapy. The therapy targets immune checkpoints, key regulators of the immune system that when stimulated can dampen the immune response to an immunologic stimulus. Some cancers can protect themselves from attack by stimulating immune checkpoint targets. Checkpoint therapy can block inhibitory checkpoints, restoring immune system function, and permitting the immune system to respond to the cancer.

Key immune checkpoint inhibitors target the molecules CTLA4, PD-1, and PD-L1. PD-1 is the transmembrane programmed cell death 1 protein (also called PDCD1 and CD279), which interacts with PD-L1 (PD-1 ligand 1, or CD274). PD-L1 on the cell surface binds to PD1 on an immune cell surface, which inhibits immune cell activity. Among PD-L1 functions is a key regulatory role on T cell activities. It appears that (cancer-mediated) upregulation of PD-L1 on the cell surface may inhibit T cells that might otherwise attack. Antibodies that bind to either PD-1 or PD-L1 and therefore block the interaction may allow the T-cells to attack the tumour.

A number of ICI therapies targeting these molecules have been approved for a wide range of uses, and more therapies and cancer targets are under investigation. Approved ICIs include ipilimumab (targeting CTLA-4); nivolumab, pembrolizumab, and cemiplimab (targeting PD-1); and atezolizumab, avelumab, and durvalumab (targeting PD-L1).

Lurbinectedin, also known as PM01183 and initially called tryptamicidin, is a synthetic tetrahydropyrrolo[4,3,2-de]quinolin-8(1H)-one alkaloid analogue with antineoplastic activity, and the subject of WO 03/014127. Lurbinectedin is a selective inhibitor of oncogenic transcription, induces DNA double-strand break generating apoptosis, and modulates the tumor microenvironment. For example, by inhibiting active transcription in tumor-associated macrophages, lurbinectedin downregulates IL-6, IL-8, CCL2, and VEGF.

The chemical structure of lurbinectedin is represented as follows:

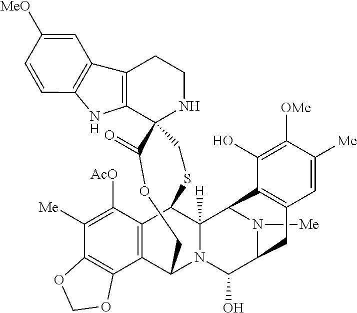 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C)OCO4]

Lurbinectedin has demonstrated highly potent in vitro activity against solid and non-solid tumour cell lines as well as significant in vivo activity in several xenografted human tumor cell lines in mice, such as those for breast, kidney and ovarian cancer. It is a selective inhibitor of the oncogenic transcription programs on which many tumours are particularly dependent. Together with its effect on cancer cells, lurbinectedin inhibits oncogenic transcription in tumour-associated macrophages, downregulating the production of cytokines that are essential for the growth of the tumour. Transcriptional addiction is an acknowledged target in those diseases, many of them lacking other actionable targets.

There is a need for further effective cancer therapies.

### SUMMARY OF THE INVENTION

The present inventors have surprisingly determined that combination therapy using lurbinectedin and an ICI may be effective in treatment of certain cancer types.

Accordingly, the present invention provides a method of treatment of a solid tumour, the method comprising administering a combination therapy of lurbinectedin and an immune checkpoint inhibitor to a patient, preferably a human patient, in need thereof, thereby treating the solid tumour.

The immune checkpoint inhibitor may comprise an immunoglobulin molecule, preferably an antibody, targeting an immune checkpoint molecule. By “targeting” is meant that the immunoglobulin molecule is an agonist of the immune checkpoint molecule, and/or that it specifically binds to the immune checkpoint molecule so as to block activation of the immune checkpoint, thereby enhancing immune function or response. The immune checkpoint molecule may be selected from CTLA-4, PD-1, and PD-L1. In preferred embodiments the immune checkpoint molecule is PD-1. In some embodiments, a plurality of immune checkpoint molecules may be targeted; for example, CTLA-4 and PD-1, or CTLA-4 and PD-L1, or CTLA-4 and PD-1 and PD-L1; preferably CTLA-4 and PD-1.

In some embodiments, the immune checkpoint inhibitor comprises a monoclonal antibody which specifically binds CTLA-4, or which specifically binds PD-1, or which specifically binds PD-L1. Examples of such monoclonal antibodies include pembrolizumab, nivolumab, ipilimumab, avelumab, atezolizumab, durvalumab, cemiplimab (REGN2810), camrelizumab (SHR1210), envafolimab (KN035), sintilimab (IBI308), spartalizumab (PDR001), tislelizumab (BGB-A317), prolgolimab (BCD-100), toripalimab (JS001), dostarlimab (TSR-042, WBP-285), tremelimumab (ticilimumab, CP-675,206).

Particularly preferred combinations include lurbinectedin and atezolizumab; lurbinectedin and pembrolizumab; lurbinectedin and nivolumab and ipilimumab; lurbinectedin and durvalumab; and lurbinectedin and dostarlimab.

In some embodiments, the immune checkpoint inhibitor comprises a peptide inhibitor of PD-1/PD-L1 interaction, or a small molecule inhibitor. Examples of such include AUNP12, CA-170, and BMS-986189.

The lurbinectedin and the immune checkpoint inhibitor may be administered concurrently, separately or sequentially. Multiple administrations of either the lurbinectedin, or the immune checkpoint inhibitor, or both, may be given. Other administration schedules may be used.

Lurbinectedin may be administered in cycles once every one to four weeks, preferably once every three weeks. A particular administration cycle is once every 21 days.

Any suitable administration route may be used, for example, subcutaneous, intravenous, intraperitoneal. Different administration routes may be used for the lurbinectedin and the immune checkpoint inhibitor. Preferably the lurbinectedin is administered by intravenous infusion; for example, 3.2 mg/m² by intravenous infusion every 21 days or three weeks, or 3.2 mg/m² by intravenous infusion over 60 minutes every 21 days or three weeks. The lurbinectedin may be administered in cycles once every one to four weeks, preferably once every three weeks. The lurbinectedin may be administered at a dose of 1 to 5 mg/m² body surface area, 1 to 2.5 mg/m² body surface area, 1 to 2 mg/m² body surface area, 2 to 3 mg/m² body surface area, about 3 mg/m² body surface area, 3 to 3.5 mg/m² body surface area, 2 to 3.2 mg/m² body surface area, 1 mg/m², 1.5 mg/m², 2 mg/m², 2.4 mg/m², 2.5 mg/m², 2.6 mg/m², or 3.2 mg/m² body surface area.

The lurbinectedin may be administered as an infusion, preferably with an infusion time of up to 24 hours, 1 to 12 hours, 1 to 6 hours and most preferably 1 hour.

The lurbinectedin may be administered in the form of a pharmaceutically acceptable salt selected from the hydrochloride, hydrobromide, hydroiodide, sulfate, nitrate, phosphate, acetate, trifluoroacetate, maleate, fumarate, citrate, oxalate, succinate, tartrate, malate, mandelate, methanesulfonate p-toluenesulfonate, sodium, potassium, calcium and ammonium salts, ethylenediamine, ethanolamine, N,N-dialkylenethanolamine, triethanolamine and basic amino acids salts.

Preferably the immune checkpoint inhibitor is administered by intravenous infusion; for example, 200 mg every 3 weeks administered as an intravenous infusion over 30 minutes.

Preferably the solid tumour is malignant. In some embodiments, the solid tumour is a carcinoma. In one embodiment of the invention, the solid tumour is selected from the group consisting of prostate cancer, breast cancer, lung cancer, colorectal cancer, melanomas, bladder cancer, brain/CNS cancer, cervical cancer, oesophageal cancer, gastric cancer, head/neck cancer, kidney cancer, liver cancer, lymphomas, ovarian cancer, pancreatic cancer, and sarcomas. For example, the solid tumour may be selected from the group consisting of cancers of the prostate gland, breast, skin, colon, lung, and urinary organs. In another embodiment, the solid tumour may be selected from the groups consisting of prostate cancer, melanomas, cervical cancer, oesophageal cancer, and head and/or neck cancer. In preferred embodiments, the solid tumour is a melanoma.

In some embodiments, the solid tumour may be a sarcoma. In some embodiments, the solid tumour may be a lymphoma.

In some embodiments, the solid tumour expresses PD-L1. In some embodiments, the method may further comprise determining whether the tumour to be treated expresses PD-L1 prior to beginning treatment. Any suitable test may be used; for example, immunohistochemistry may be used to detect PD-L1 expression on the cell surface of tumour cells.

The treatment may result in one or more of the following outcomes: reduction in tumour size; delay in growth of tumour; prolongation of life of the patient; remission. These outcomes may be in comparison to a control subject (or hypothetical control subject) not given the treatment, or given an alternative treatment.

The above features also apply to the following aspects of the invention, unless otherwise noted.

A further aspect of the present invention provides a method of prolonging survival of a patient having a solid tumour, the method comprising administering a combination therapy of lurbinectedin and an immune checkpoint inhibitor to a patient in need thereof, thereby prolonging survival of the patient.

Also provided is a method of delaying disease progression of a solid tumour in a patient, the method comprising administering a combination therapy of lurbinectedin and an immune checkpoint inhibitor to a patient in need thereof, thereby delaying disease progression of the solid tumour.

Yet further provided is a method of reducing or delaying growth of a solid tumour, the method comprising administering a combination therapy of lurbinectedin and an immune checkpoint inhibitor to a patient in need thereof, thereby reducing or delaying growth of the solid tumour.

A still further aspect of the invention provides a method of selecting a patient having a solid tumour for combination therapy, the method comprising determining whether the solid tumour expresses PD-L1, and if so, selecting the patient for combination therapy wherein the combination therapy comprises administering a combination therapy of lurbinectedin and an immune checkpoint inhibitor. Preferably the immune checkpoint inhibitor comprises an immunoglobulin which targets PD-1 or PD-L1. The method may further comprise providing said combination therapy to the patient.

Also provided by the present invention is use of lurbinectedin in the manufacture of a medicament for the treatment of a solid tumour, wherein said treatment comprises administering a combination therapy of lurbinectedin and an immune checkpoint inhibitor to a patient in need thereof.

The invention also provides use of an immune checkpoint inhibitor in the manufacture of a medicament for the treatment of a solid tumour, wherein said treatment comprises administering a combination therapy of lurbinectedin and an immune checkpoint inhibitor to a patient in need thereof.

Yet further provided is use of lurbinectedin and an immune checkpoint inhibitor in the manufacture of a medicament for the treatment of a solid tumour, wherein said treatment comprises administering a combination therapy of lurbinectedin and an immune checkpoint inhibitor to a patient in need thereof.

The invention further provides lurbinectedin for use in a method of treatment of a solid tumour, wherein said treatment comprises administering a combination therapy of lurbinectedin and an immune checkpoint inhibitor to a patient in need thereof.

Also provided is an immune checkpoint inhibitor for use in a method of treatment of a solid tumour, wherein said treatment comprises administering a combination therapy of lurbinectedin and an immune checkpoint inhibitor to a patient in need thereof.

The invention further provides lurbinectedin and an immune checkpoint inhibitor for use in a method of treatment of a solid tumour, wherein said treatment comprises administering a combination therapy of lurbinectedin and an immune checkpoint inhibitor to a patient in need thereof.

Dosage forms, pharmaceutical packages and preparations, and kits of parts are also provided by the invention. These may comprise lurbinectedin and/or an immune checkpoint inhibitor packaged for use in a method of treatment of a solid tumour, wherein said treatment comprises administering a combination therapy of lurbinectedin and an immune checkpoint inhibitor to a patient in need thereof. The dosage forms, packages, preparations and kits may further comprise instructions for providing treatment to a patient.

### BRIEF DESCRIPTION OF THE FIGURES

FIG. 1. Immunogenic cell death assessment in solid tumours.

(a) Human osteosarcoma U2OS (a), human breast cancer HCC70 (b) human colon cancer HT29 cells (c) and murine methylcholantrene-induced fibrosarcoma MCA205 cells (d) were treated with lurbinectedin (Lurbi, 1 nM, 10 nM, 100 nM and 1 μM) for the indicated times. Subsequently, the cells were stained with 1 μM Hoechst 33342 and 1 μM propidium iodide and assessed for the loss of viability by automated image acquisition. Images were segmented, cellular debris was excluded and the number of cells with normal nuclear morphology was enumerated. Cells stably expressing CALR-GFP were treated as above. Following the cells were fixed with 3.7% of PFA, stained with 1 μM Hoechst 33342 and assessed by automated image acquisition. Images were segmented, cellular debris was excluded and CALR-GFP granularity (a surrogate marker of CALR exposure) was evaluated in the cytoplasmic region of cells with normal nuclear morphology. Wild type cells were treated as above and then assessed for cytoplasmic quinacrine granularity (after staining with the ATP-sensitive dye quinacrine together with Hoechst 33342) by automated image acquisition, segmentation and analysis. Cells stably expressing HMGB1-GFP were treated as above and then assessed for nuclear HMGB1-GFP fluorescence intensity. The cells were fixed and stained with Hoechst 33342 and images were acquired, segmented and analyzed. WT cells were treated as above and following the media was changed and the cells were incubated for 48 hours before the supernatant was used to treat MX1-GFP biosensor cells for additional 48 hours. The cells were fixed and stained with Hoechst 33342 before type 1 IFN responses were monitored by means of automated microscopy as an increase in GFP fluorescence intensity. Mitoxantrone (MTX, 1 and 3 μM) was used as a positive control. The means of quadruplicate assessments and p-values are depicted as heat maps. (*p<0.01; **p<0.005; ***p<0.001, two-tailed Student's t test).

FIG. 2. Traits of immunogenic cell death.

Human osteosarcoma U2OS cells were treated with 10, 50 or 100 nM lurbinectedin (Lurbi) for 6 hours. Thapsigargin (Thaps, 3 μM) was used as a positive control. The cells were fixed with 3.7% PFA and DNA was stained with 1 μM Hoechst 33342. Following the phosphorylation of the eukaryotic translation initiation factor 2 alpha (eIF2a) was assessed with phosphoneoepitope-specific antibody and was monitored by means of automated microscopy as an increase in cytoplasmic fluorescence intensity. (a,b) The level of transcription was measured in U2OS cell treated as above with Lurbi. The transcription inhibitor actinomycin D (ActD) was used as a control. The cells were fixed as above and following the colocalization of nucleolin and fibrillarin was assessed as an indicator for transcriptional activity (c,d) Scale bar equals 10 μm and bar charts depict mean values±SD of quadruplicate assessments (*p<0.01; *p<0.001, two-tailed Student's t test).

FIG. 3. Anticancer vaccination efficacy of lurbinectedin-treated cells.

MCA205 cells treated for 20 h with 1 μM lurbinectedin were inoculated subcutaneously (s.c.) into immunocompetent C57BL/6 mice, which were rechallenged 7 days later s.c. with living cells of the same type. The tumour growth was measured until endpoints were reached and overall survival was evaluated regularly for the following 30 days (n=6). (*p<. 01, two-tailed Student's t test, compared to all other groups). Data were analyzed with TumGrowth.

FIG. 4. Therapeutic efficacy of lurbinectedin in immunocompetent and immunodeficient hosts.

Live MCA205 cells were injected subcutaneously (s.c.) into immunocompetent C57BL/6 mice or immunodeficient nu/nu mice as depicted in the scheme in (a) When tumours became palpable, mice were intravenously (i.v.) injected with 0.14 mg/Kg lurbinectedin (on day 1, 7 and 14). Tumour growth was assessed regularly for the following 30 days. Data is depicted as tumour growth curves (b,d) and overall survival plots (c,e). Data were analyzed with TumGrowth.

FIG. 5. Sequential lurbinectedin treatment with double immune checkpoint blockade exhibits systemic antitumor immunity

C57BL/6 mice were inoculated subcutaneously (s.c.) with murine fibrosarcoma MCA205. Palpable tumours were treated with sequential intravenous (i.v.) injections of 0.14 mg/Kg lurbinectedin (Lurbi) as indicated in (a). Single- or double-immune checkpoint blockade was mounted by sequential intraperitoneal (i.p.) injections of monoclonal antibodies targeting CTLA-4 or PD-1 at day 6, 9 and 12 post treatment and tumour growth (b,c) and overall survival (d,e) were assessed regularly for the following 30 days. (f,g) The generation of immunological memory was assessed in cured animals by rechallenge with MCA205 and TC-1. Naïve animals were used as controls. Individual tumour growth curves are depicted. Data were analyzed with TumGrowth.

FIG. 6. Lurbinectedin retards the growth of spontaneous tumours.

Medroxyprogesterone acetate (MPA) pellets (50 mg, 90-day release) were implanted subcutaneously into the interscapular area of immunocompetent C57BL/6 mice. Then the animals received 1 mg dimethylbenzantracene (DMBA) administered by oral gavage 6× during 7 weeks. When spontaneous tumours became palpable mice were randomly assigned to receive 0.14 mg/Kg lurbinectedin (Lurbi) alone or in combination with double immune checkpoint blockade with monoclonal antibodies targeting CTLA-4 and PD-1 at day 6, 9 and 12 post treatment (a). The tumour area and overall survival were measured regularly until ethical endpoints were reached (b,c,d). Data were analyzed with TumGrowth (https://github.com/kroemerlab).

### DETAILED DESCRIPTION OF THE INVENTION

In the present application, a number of general terms and phrases are used, which should be interpreted as follows.

The term “treating”, as used herein, unless otherwise indicated, means reversing, attenuating, alleviating or inhibiting the progress of the disease or condition to which such term applies, or one or more symptoms of such disorder or condition. The term “treatment”, as used herein, unless otherwise indicated, refers to the act of treating as “treating” is defined immediately above.

“Patient” includes humans, non-human mammals (e.g., dogs, cats, rabbits, cattle, horses, sheep, goats, swine, deer, and the like) and non-mammals (e.g., birds, and the like).

Lurbinectedin is a synthetic alkaloid, having the following structure:

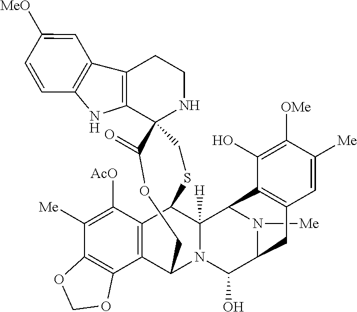 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C)OCO4]

Information regarding its mechanism of action and in vivo efficacy can be found in 100th AACR Annual Meeting, Apr. 18-22, 2009, Denver, CO, Abstract Nr. 2679 and Abstract Nr. 4525; Leal J F M et. al. Br. J. Pharmacol. 2010, 161, 1099-1110; and Belgiovine, C et al. Br. J. Cancer, 2017; 117 (5): 628-638;

Further information regarding the clinical development of PM01183 (lurbinectedin) can be found in:
Elez, M E. et. al. Clin. Cancer Res. 2014, 20 (8), 2205-2214;
50th ASCO Annual Meeting, May 30-Jun. 3, 2014, Chicago, IL, Abstract 5505;
26th EORTC-NCI-AACR Symposium on Molecular Targets and Cancer Therapeutics; Nov. 18-21, 2014, Barcelona, Spain, published in Eur. J. Cancer 2014, 50 (Suppl. 6), pages 13-14, Abs. No. 23.
51th ASCO Annual Meeting, May 29-Jun. 2, 2015, Chicago, IL, Abstract No. TPS2604 and Abstract Nr. 7509, published in J. Clin. Oncol. 33, 2015 (suppl);
54th ASCO Annual Meeting, Jun. 1-5, 2018, Chicago, IL, Abstract No. 11519, published in J. Clin. Oncol. 36, 2018 (suppl);
Cruz, C. et al. J. Clin. Oncol. 2018; in press 1-21;
54th ASCO Annual Meeting, Jun. 1-5, 2018, Chicago, IL, Abstract No. 8570, published in J. Clin. Oncol. 36, 2018 (suppl);

The term “lurbinectedin” is intended here to cover any pharmaceutically acceptable salt, ester, solvate, hydrate, prodrug, or any other compound which, upon administration to the patient is capable of providing (directly or indirectly) the compound as described herein. However, it will be appreciated that non-pharmaceutically acceptable salts also fall within the scope of the invention since those may be useful in the preparation of pharmaceutically acceptable salts. The preparation of salts can be carried out by methods known in the art.

For instance, pharmaceutically acceptable salts of the compounds provided herein are synthesized from the parent compounds, which contain a basic or acidic moiety, by conventional chemical methods. Generally, such salts are, for example, prepared by reacting the free acid or base of these compounds with a stoichiometric amount of the appropriate base or acid in water or in an organic solvent or in a mixture of both. Generally, nonaqueous media like ether, ethyl acetate, ethanol, 2-propanol or acetonitrile are preferred. Examples of the acid addition salts include mineral acid addition salts such as, for example, hydrochloride, hydrobromide, hydroiodide, sulfate, nitrate, phosphate, and organic acid addition salts such as, for example, acetate, trifluoroacetate, maleate, fumarate, citrate, oxalate, succinate, tartrate, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic amino acids salts.

Any compound that is a prodrug of lurbinectedin is within the scope and spirit of the invention. The term “prodrug” is used in its broadest sense and encompasses those derivatives that are converted in vivo to PM01183. The prodrug can hydrolyze, oxidize, or otherwise react under biological conditions to provide PM01183. Examples of prodrugs include, but are not limited to, derivatives and metabolites of PM01183 that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Prodrugs can typically be prepared using well-known methods, such as those described by Burger in “Medicinal Chemistry and Drug Discovery” 6th ed. (Donald J. Abraham ed., 2001, Wiley) and “Design and Applications of Prodrugs” (H. Bundgaard ed., 1985, Harwood Academic Publishers).

In addition, any drug referred to herein may be in crystalline or amorphous form either as free compounds or as solvates (e.g. hydrates) and it is intended that all forms are within the scope of the present invention. Methods of solvation are generally known within the art.

Moreover, lurbinectedin for use in accordance with the present invention may be prepared following the synthetic process such as the one disclosed in WO 03/014127, which is incorporated herein by reference.

In a preferred embodiment of the combination of the present invention, the molar ratio of lurbinectedin or a pharmaceutically acceptable salt or stereoisomer thereof to immune checkpoint inhibitor in said combination is from 1:1000 to 1000:1. Further molar ratios include 1:700 to 700:1, 1:500 to 500:1, 1:300 to 300:1, 1:100 to 100:1, and 1:50 to 50:1.

Pharmaceutical compositions comprising lurbinectedin or a pharmaceutically acceptable salt or ester thereof, and a pharmaceutically acceptable carrier may be formulated according to the chosen route of administration. Examples of the administration form include without limitation oral, topical, parenteral, sublingual, rectal, vaginal, ocular and intranasal. Parenteral administration includes subcutaneous injections, intravenous, intramuscular, intrasternal injection or infusion techniques. Preferably the compositions are administered parenterally. Pharmaceutical compositions can be formulated so as to allow a compound to be bioavailable upon administration of the composition to an animal, preferably human. Compositions can take the form of one or more dosage units, where for example, a tablet can be a single dosage unit, and a container of a compound may contain the compound in liquid or in aerosol form and may hold a single or a plurality of dosage units.

The pharmaceutically acceptable carrier or vehicle can be particulate, so that the compositions are, for example, in tablet or powder form. The carrier(s) can be liquid, with the compositions being, for example, an oral syrup or injectable liquid. In addition, the carrier(s) can be gaseous, or liquid so as to provide an aerosol composition useful in, for example inhalatory administration. Powders may also be used for inhalation dosage forms. The term “carrier” refers to a diluent, adjuvant or excipient, with which the compound according to the present invention is administered. Such pharmaceutical carriers can be liquids, such as water and oils including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, disaccharides, and the like. In addition, auxiliary, stabilizing, thickening, lubricating and coloring agents can be used. In one embodiment, when administered to an animal, the compounds and compositions and pharmaceutically acceptable carriers are sterile. Water is a preferred carrier when the compounds are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more for the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agent such as sucrose or saccharin; a flavoring agent such as peppermint, methyl salicylate or orange flavoring; and a coloring agent.

When the composition is in the form of a capsule (e.g. a gelatin capsule), it can contain, in addition to materials of the above type, a liquid carrier such as polyethylene glycol, cyclodextrins or a fatty oil.

The composition can be in the form of a liquid, e.g. an elixir, syrup, solution, emulsion or suspension. The liquid can be useful for oral administration or for delivery by injection. When intended for oral administration, a composition can comprise one or more of a sweetening agent, preservatives, dye/colorant and flavour enhancer. In a composition for administration by injection, one or more of a surfactant, preservative, wetting agent, dispersing agent, suspending agent, buffer, stabilizer and isotonic agent can also be included.

The preferred route of administration is parenteral administration including, but not limited to, intradermal, intramuscular, intraperitoneal, intravenous, subcutaneous, intranasal, epidural, intracerebral, intraventricular, intrathecal, intravaginal or transdermal. The preferred mode of administration is left to the discretion of the practitioner, and will depend in part upon the site of the medical condition. In a more preferred embodiment, the compounds according to the present invention are administered intravenously. Infusion times of up to 24 hours are preferred to be used, more preferably 1 to 12 hours, with 1 to 6 hours being most preferred. Short infusion times which allow treatment to be carried out without an overnight stay in a hospital are especially desirable. However, infusion may be 12 to 24 hours or even longer if required. Infusion may be carried out at suitable intervals of, for example, 1 to 4 weeks, preferably once every three weeks.

Liquid compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono or diglycerides, polyethylene glycols, glycerin, or other solvents; antibacterial agents such as benzyl alcohol or methyl paraben; and agents for the adjustment of tonicity such as sodium chloride or dextrose. A parenteral composition can be enclosed in an ampoule, a disposable syringe or a multiple-dose vial made of glass, plastic or other material. Physiological saline is a preferred adjuvant.

The compositions comprise an effective amount of a lurbinectedin and/or an immune checkpoint inhibitor such that a suitable dosage will be obtained. The correct dosage will vary according to the particular formulation, the mode of application, and its particular site and host. Other factors like age, body weight, sex, diet, time of administration, rate of excretion, condition of the host, drug combinations, reaction sensitivities and severity of the disease should be taken into account. Administration can be carried out continuously or periodically within the maximum tolerated dose.

The dose will be selected according to the dosing schedule, having regard to the existing data on preferred administration routes and dosages for each compound.

In specific embodiments, it can be desirable to administer lurbinectedin or an immune checkpoint inhibitor locally to the area in need of treatment. In one embodiment, administration can be by direct injection at the site (or former site) of a cancer, tumour or neoplastic or pre-neoplastic tissue.

Pulmonary administration can also be employed, e.g. by use of an inhaler or nebulizer, and formulation with an aerosolizing agent, or via perfusion in a fluorocarbon or synthetic pulmonary surfactant. In certain embodiments, lurbinectedin can be formulated as a suppository, with traditional binders and carriers such as triglycerides.

The present compositions can take the form of solutions, suspensions, emulsions, tablets, pills, pellets, capsules, capsules containing liquids, powders, sustained-release formulations, suppositories, emulsions, aerosols, sprays, suspensions, or any other form suitable for use. Other examples of suitable pharmaceutical carriers are described in “Remington's Pharmaceutical Sciences” by E. W. Martin.

The pharmaceutical compositions can be prepared using methodology well known in the pharmaceutical art. For example, a composition intended to be administered by injection can be prepared by combining lurbinectedin with water, or other physiologically suitable diluent, such as phosphate buffered saline, so as to form a solution. A surfactant can be added to facilitate the formation of a homogeneous solution or suspension.

Preferred compositions comprising lurbinectedin may invention include:
Pharmaceutical compositions comprising lurbinectedin and a disaccharide. Particularly preferred disaccharides are selected from lactose, trehalose, sucrose, maltose, isomaltose, cellobiose, isosaccharose, isotrehalose, turanose, melibiose, gentiobiose, and mixtures thereof.
Lyophilised pharmaceutical compositions comprising lurbinectedin and a disaccharide. Particularly preferred disaccharides are selected from lactose, trehalose, sucrose, maltose, isomaltose, cellobiose, isosaccharose, isotrehalose, turanose, melibiose, gentiobiose, and mixtures thereof.

The ratio of lurbinectedin to the disaccharide in embodiments of the present invention is determined according to the solubility of the disaccharide and, when the formulation is freeze dried, also according to the freeze-dryability of the disaccharide. It is envisaged that this lurbinectedin:disaccharide ratio (w/w) can be about 1:10 in some embodiments, about 1:20 in other embodiments, about 1:50 in still other embodiments. It is envisaged that other embodiments have such ratios in the range from about 1:5 to about 1:500, and still further embodiments have such ratios in the range from about 1:10 to about 1:500.

The composition comprising lurbinectedin may be lyophilized. The composition comprising lurbinectedin is usually presented in a vial which contains a specified amount of such compound.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The invention will now be described further with reference to the following example.

### EXAMPLE

### Introduction

Primary or transplantable tumours react to anthracycline-based chemotherapy with durable response in syngeneic immunocompetent mice yet fail to do so in immunodeficient hosts (1-3). Consistently, retrospective clinical studies in patients with solid tumours subjected to chemotherapy showed that severe lymphopenia negatively affects prognosis, (4,5) which points to the fact that chemotherapy-elicited anticancer immunity plays a critical role for the outcome of anticancer therapy. (6,7) Based on these findings, (1-3) we introduced the hypothesis that some chemotherapeutic agents can induce immunogenic cell death (ICD) in tumours and convert them into a therapeutic vaccine, hence stimulating an immune response that can control residual cancer cells.

Selected chemotherapeutics such as anthracyclines and oxaliplatin are able to induce ICD (1-3) while many other antineoplastic agents including cisplatin and mitomycin C fail to do so. Cancer cells undergoing ICD can evoke anticancer immunity and protect against a subsequent challenge with living cells exhibiting the same antigenic profile in mice (1-3) or elicit anticancer immune responses during chemotherapy in patients. (8) Distinctive properties of immunogenic cell death include the exposure of calreticulin (CALR) at the cytoplasmic surface, (3,8,9) the autophagy-dependent liberation of ATP from stressed and dying cells, (10,11) the cell death-associated exodus of nuclear high mobility group box 1 (HMGB1) (12,13) and the stimulation of an autocrine or paracrine type-1 interferon response. (14) CALR serves as a de novo uptake signal and stimulates the engulfment of dying cancer cells by dendritic cells (DCs). (3) HMGB1 binds to toll-like receptor-4 (TLR4) entities on DC, eliciting MYD88-dependent signaling that facilitates tumor antigen processing. (3,15) ATP ligates purinergic receptors of the P2X type and thus activates the NLRP3 inflammasome to stimulate the production of interleukin-1β (IL-1B) by DC and eventually interferon-γ (IFNγ) by CD8+ cytotoxic T lymphocytes (CTL). (10,16) The sum of danger associated molecular patterns (DAMP) emitted during ICD is necessary to generate anticancer immunity, thus tumours growing in Tlr4−/−, P2rx7−/−, Myd88−/−, Nlrp3−/−, Il1r−/−, Ifnγ−/−, Ifnγr−/−, Fpr1−/−, athymic or CD8+ T cell-depleted mice fail to respond to immunogenic chemotherapeutic regimens. Loss-of-function mutations of FPR1, P2RX7 or

TLR4 in breast cancer are negatively correlated with clinical response to adjuvant chemotherapy with anthracyclines. (3,10,13,14,17-19) These results imply the obligate contribution of anticancer immune responses to the success of ICD-inducing chemotherapies. Here, we investigated the capacity of lurbinectedin to stimulate the emission of immunogenic DAMPs and tested anticancer immune responses in three experimental in vivo models. Our results support the contention that lurbinectedin causes immunogenic cell death in tumours and creates anticancer immunity.

### Results and Discussion

### Emission of Immunogenic Signals by Lurbinectedin

The known parameters determining ICD are the translocation of CALR to the surface of the plasma membrane, the autophagy-dependent liberation of ATP and the release of the non-histone binding protein HMGB1, which occur before, during and after apoptosis, respectively. The production of type I interferons (IFNs) has been added to the list of ICD hallmarks as it controls autocrine or paracrine circuits that underlie cancer immunosurveillance.

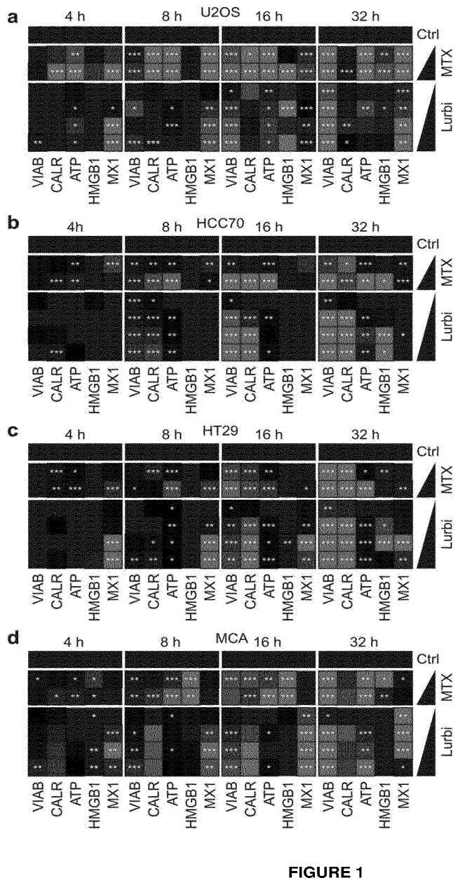

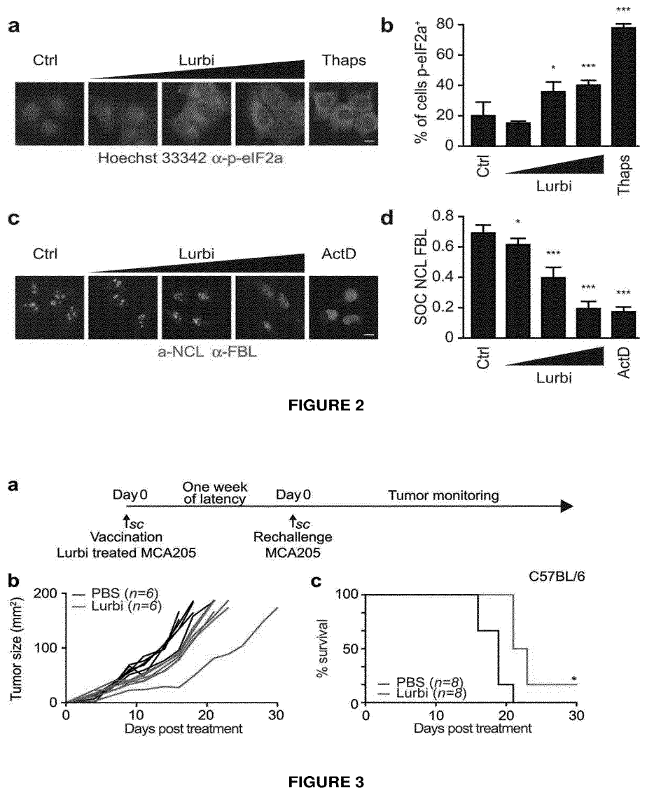

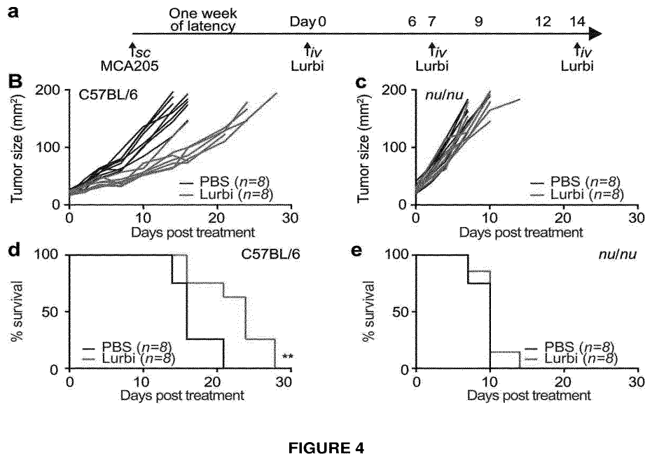

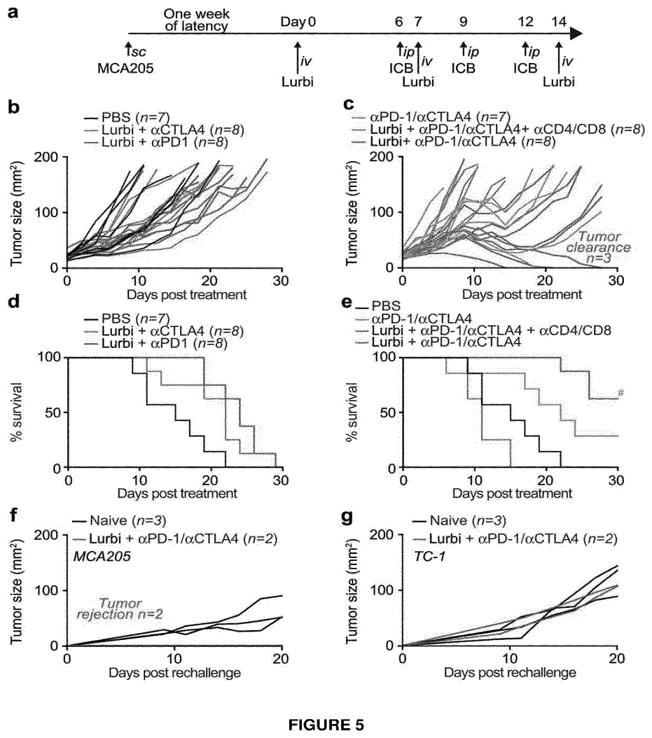

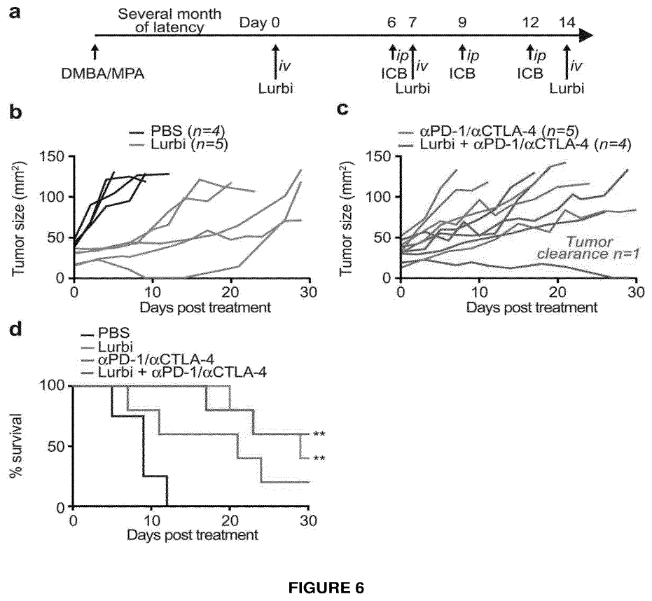

In a systematic screening campaign, the capacity of lurbinectedin to induce immunogenic cell death in cancer cells was assessed in human osteosarcoma U2OS cells stably expressing fluorescent biosensors for the detection of CALR-relocation (as a surrogate marker for CALR surface exposure), HMGB1 release and Type I IFN responses together with U2OS WT cells stained with the ATP-sensitive dye quinacrine. ICD-related parameters were measured at 4, 8, 16 and 32 hours post exposure to lurbinectedin from 1 nM to 1 μM by robotized epifluorescence microscopy followed by automated image analysis (FIG. 1). The induction of cell death was evaluated based on changes in the nuclear morphology visualized by means of the DNA intercalating dye Hoechst 33342. Lurbinectedin caused a dose- and time-dependent drop in cellular viability comparable to mitoxantrone (MTX) that was used at 1 and 3 μM as a positive control throughout all experiments. The translocation of a CALR-GFP (green fluorescent protein) fusion protein from the perinuclear ER to the cellular periphery was measured by assessing cytoplasmic “granularity” (see Materials and Methods) as an indicator for the formation of CALR-containing vesicles and as a surrogate marker for CALR exposure. Lurbinectedin, similar to MTX, induced a time- and dose-dependent increase in CALR-granularity as compared to untreated controls. The reduction of intracellular ATP (as an indicator for ATP release) was assessed by measuring the decrease in the cytoplasmic granularity of ATP containing vesicles stained with the fluorescent probe quinacrine. As compared to untreated controls a significant decrease in ATP signal similar to MTX was detectable for lurbinectedin. The effect was dose-dependent and decreased over time in line with the fragile nature of the metabolite. HMGB1 release was detected as a loss in the nuclear fluorescence of an HMGB1-GFP chimera. A significant decrease in nuclear GFP signal was detected for MTX and lurbinectedin at medium to late time points. Type I interferon (IFN) production was measured using U2OS biosensor cells stably expressing a GFP under the control of the MX1 (a Type I IFN response gene) promoter. To this aim the supernatant of U2OS cells following treatment and additional 48 hours incubation with fresh media was used to treat the biosensor cells. Following the type 1 IFN response was monitored by means an increase in GFP fluorescence intensity. A significant increase in de novo GFP signal intensity was detected for both lurbinectedin and MTX throughout all time points (FIG. 1(a)). Similar results were obtained when the approach was repeated in human breast cancer HCC70 cells (FIG. 1(b)), human colon carcinoma HT29 (FIG. 1(c)) and mouse fibrosarcoma MCA205 cells (FIG. 1(d)). Next, we investigated the capacity of lurbinectedin to activate two additional characteristics of common ICD inducers, the phosphorylation of the eukaryotic translation initiation factor 2 alpha (eIF2α) and the inhibition of general transcription. Indeed, lurbinectedin led to a dose-dependent phosphorylation of eIF2α monitored by fluorescence microscopy upon immunostaining with a phosphoneoepitope-specific antibody (FIG. 2(a,b)). Lurbinectedin also inhibited mRNA transcription at a level comparable to a known transcription-inhibitor, as assessed by visualizing the dissociation of nucleolin and fibrillarin by microscopy (FIG. 2(b,c)), an accepted proxy of suppressed transcription. (21) Lurbinectedin holds many of the described in vitro parameters of ICD, thus qualifying for further in vivo investigations in immunocompetent animals, which remains the gold standard assay for the determination of ICD-mediated anticancer immunity.

### Anticancer Immunity Induced by Lurbinectedin

In order to assess the capacity of lurbinectedin to stimulate anticancer immunity in a monotherapeutic approach and to convert tumour cells into a therapeutic vaccine we exposed murine fibrosarcoma cells to the drug in vitro (in conditions previously established to induce a sufficient amplitude of cell death) and then injected the dying cancer cells into syngeneic immunocompetent mice. One week later, the animals were re-challenged injecting live tumour cells of the same kind into the opposite flank, (FIG. 3(a)). In this setting, a decrease of tumour growth can be interpreted as sign of a productive anticancer immune response. Indeed, lurbinectedin-treated cells significantly reduced tumour growth (p=0.0094) (FIG. 3(b)) and led to an increase in overall survival (FIG. 3(c)). As compared to know ICD inducers (1-3) the vaccination effects observed here were rather limited yet statistically significant. Next we evaluated the effect of lurbinectedin on established cancers growing on immunocompetent or immunodeficient mice. MCA 205 tumours were implanted subcutaneously on immunocompetent C57BL/6 as well as in athymic nu/nu mice. When the tumours became palpable, the animals were treated with three consecutive intravenous injections of 0.18 mg/kg lurbinectedin on day 1, 7 and 14. (FIG. 4(a)). The treatment with lurbinectedin had significant therapeutic benefit in immunocompetent animals. The tumour growth was significantly reduced as compared to control animals (p<0.0001) (FIG. 4(b)) and overall survival was increased (FIG. 4(c)). This effect was exclusively observed when tumours grew on immunocompetent mice, yet was lost when the tumours proliferated on athymic (nu/nu) mice (FIG. 4(d,e)). These results underscore the obligate contribution of the immune system to the chemotherapeutic activity of lurbinectedin.

### Combinatorial Effects of Lurbinectedin and αPD-1/αCTLA-4 Double Immune Checkpoint Blockade

Given the capacity of lurbinectedin to induce immune-dependent anticancer effects on established tumours, we investigated whether this agent could sensitize cancers to therapy with immune checkpoint blockers targeting CTLA-4 or PD-1. For this, established MCA205 fibrosarcomas were treated with Lurbinectedin as before and subjected to immunotherapy with antibodies specific for CTLA-4, PD-1 or a combination of both on day 6, 9 and 12, when the anticancer immune response in the tumour peaks (FIG. 5(a)). Tumor monitoring led to the deduction that the most efficient therapeutic regimen was a combination of all three anticancer agents (lurbinectedin, αCTLA-4 and αPD-1). Single ICB therapies are also shown to be effective (FIG. 5(b-e)). The combination of lurbinectedin with αCTLA-4/αPD-1 dual checkpoint blockade in tumour-bearing animals significantly extended life expectancy and, moreover, led to tumour clearance in 3/8 mice in the time frame of the experiment (FIG. 5(e)). The effect of lurbinectedin with αCTLA-4/αPD-1 dual checkpoint blockade was abrogated in conditions in which CD4+ and CD8+ cytotoxic T lymphocytes (CTLs) were depleted. Mice that had been rendered tumour-free for more than 50 days rejected tumours upon rechallenge with the same cancer cell type from which they had been cured (MCA205), yet developed cancers when rechallenged with TC1 tumour cells (FIG. 5(f,g)). Thus, mice that had been cured by a combination of systemic lurbinectedin-based chemotherapy and immunotherapy had established a specific anticancer immune response that generated immunological memory.

### Lurbinectedin Retards the Growth of Carcinogen-Induced and Spontaneous Breast Cancer

To explore the potential lurbinectedin for the therapy of breast cancer, we took advantage of a hormone/carcinogen induced breast cancer model activated by the continuous stimulation of progesterone receptors by medroxyprogesterone acetate (MPA) and the repeated exposure to the DNA-damaging agent dimethylbenzantracene (DMBA). This induced model of breast cancer is known to be modulated by the immune system. (22) We treated mice with palpable MPA/DMBA-induced tumours by systemic injection with lurbinectedin alone or in combination with double immune checkpoint blockade neutralizing CTLA-4 and PD-1 (FIG. 6(a)). Both interventions significantly reduced tumour growth and increased overall survival. However, only the combination with αCTLA-4/αPD-1 yielded tumour clearance in the time frame of the experiment (FIG. 6(b-d)).

### Concluding Remarks

The results of this study suggest that lurbinectedin efficiently induces cell death in a broad panel of solid tumours. This procedure likely does not only cause the cells to succumb to disintegration but rather triggers traits of immunogenic cell death, including the phosphorylation of eIF2a and the release of danger associated molecular patterns (DAMPs). Irrespective of the exact molecular mechanisms accounting for these effects, there are a number of evidences advocating for lurbinectedin-triggered cancer-specific immunogenicity. Thus, animals that had been cured by lurbinectedin from established cancers became resistance to rechallenge with the same cancer type. The therapeutic effect of lurbinectedin was neutralized in conditions in which either the host was immunocompromised or T-cell had been depleted. Furthermore, the recapitulation in a heterogeneous spontaneous tumour model of effects that were previously observed in homogenous transplanted tumours indicates that the results presented here hold a high translational value.

Altogether, these results convincingly demonstrate that lurbinectedin mediated immunochemotherapy may be advantageously combined with clinically established immune checkpoint blockade regimens.

### Materials & Methods

### Cell Culture and Chemicals

All media and cell culture supplements were from Thermo Fisher Scientific (Carlsbad, CA, US). Lurbinectedin was provided by PharmaMar (Madrid, Spain). Cell culture plastics and consumables were purchased from Greiner Bio-One (Kremsmünster, Austria). Human osteosarcoma U2OS cells previously genetically altered as described earlier, 23 murine methylcholanthrene-induced fibrosarcoma MCA-205 cells and murine lung cancer TC-1 cells were cultured in Glutamax®-containing DMEM medium supplemented with 10% fetal bovine serum (FBS), and 10 mM HEPES. Cells were cultured in a temperature-controlled environment at 37° C. with a humidified atmosphere containing 5% CO2.

### Automated Image Acquisition and Analysis

One day before the experiment 5×10³ cells were seeded in 96-well μClear imaging plates (Greiner BioOne) and let adhere under standard culture conditions. The following day cells were treated with lurbinectedin at 0.001, 0.01, 0.1 and 1 μM for 4, 8, 16 or 32 hours. Then cells were fixed with 3.7% formaldehyde supplemented with 1 μg/ml Hoechst 33342 for 30 min at RT. The fixative was changed to PBS and the plates were analyzed by automated microscopy. For the detection of ATP enriched vesicles, the cells were labeled after 4, 8, 16 or 32 hours of incubation with the fluorescent dye quinacrine (as described before (23)). In short, cells were incubated with 5 μm quinacrine and 1 μg/ml Hoechst 33342 in Krebs-Ringer solution (125 mM NaCl, 5 mM KCl, 1 mM MgSO₄, 0.7 mM KH₂PO₄, 2 mm CaCl₂), 6 mM glucose and 25 mM Hepes, pH 7.4) for 30 minutes at 37° C. Thereafter, cells were rinsed with Krebs-Ringer and viable cells were microscopically examined. For automated fluorescence microscopy a robot-assisted Molecular Devices IXM XL Biolmager (Molecular Devices, Sunnyvale, CA, USA) equipped with SpectraX light source (Lumencor, Beaverton, OR, USA), adequate excitation and emission filters (Semrock, Rochester, NY, USA) and a 16-bit monochromes sCMOS PCO.edge 5.5 camera (PCO Kelheim, Germany) and a 20× PlanAPO objective (Nikon, Tokyo, Japan) was used to acquire a minimum of 9 view fields, followed by automated image processing with the custom module editor within the MetaXpress software (Molecular Devices). Depending on the utilized biosensor cell line the primary region of interest (ROI) was defined by a polygon mask around the nucleus allowing for the enumeration of cells, the detection of morphological alterations of the nucleus and nuclear fluorescence intensity. Cellular debris was excluded from the analysis and secondary cytoplasmic ROIs were used for the quantification of CALR-GFP or quinacrine containing vesicles. For the latter, the images were segmented and analyzed for GFP granularity by comparing the standard deviation of the mean fluorescence intensity of groups of adjacent pixels (coefficient of variation) within the cytoplasm of each cell to the mean fluorescence intensity in the same ROI using the MetaXpress software (Molecular Devices).

### In Vivo Experimentation

Six- to eight-week-old female wild-type C57BL/6 and nu/nu mice were obtained from Envigo France (Huntingdon, UK) and were kept in the animal facility at the Gustave Roussy Campus Cancer in a specific pathogen-free and temperature-controlled environment with 12 h day, 12 h night cycles and received food and water ad libitum. Animal experiments were conducted in compliance with the EU Directive 63/2010 and protocols 2013_094A and were approved by the Ethical Committee of the Gustave Roussy Campus Cancer (CEEA IRCIV/IGR no. 26, registered at the French Ministry of Research). As described, (24,25) MCA205 tumours were established in C57BL/6 mice by subcutaneously (s.c.) injection of 5×10⁵ cells. When tumours became palpable, 0.18 mg/Kg lurbinectedin was injected sequentially once a week intravenously into the tail vein and animal well-being and tumour growth were monitored. A total of 0.5 mg of anti-CD8 (clone 2.43 BioXCell BE 0061) and anti-CD4 (clone GK1.5 BioXCell BE 0003-1) intraperitoneal (i.p.) injections were repeated every 7 days to assure the complete depletion of both T cell populations during the whole experiment. Mice were sacrificed when tumour size reached end-point or signs of obvious discomfort associated to the treatment were observed following the EU Directive 63/2010 and our Ethical Committee advice. Tumour-free animals were kept for more than 30 days before testing the generation of immunological memory by s.c. rechallenge with 5×10⁵ TC-1 in one flank and 5×10⁵ MCA205 cells injected in the contralateral flank. Animals were monitored and tumour growth documented regularly until end-points were reached. Statistical analysis was performed by applying 2-way ANOVA analysis followed by Bonferroni's test comparing to control conditions (*p<0.05, **p<0.01 and *p<0.001). Murine fibrosarcoma MCA205 cells were incubated with 1 μM lurbinectedin for 24 h, resulting in approximately 70% cell death. For vaccination experiments, 3×10⁵ dying MCA205 cells were inoculated s.c. into the left flanks of six-week-old female C57BL/6 mice. Seven to ten days later, animals were re-challenged in the opposite flank with 3×10⁵ living MCA205 cells, and tumour growth and incidence were monitored. Six-week-old female C57BL/6 mice (n=12 per group) underwent surgical implantation of slow-release medroxyprogesterone acetate (MPA) pellets (50 mg, 90-day release; Innovative Research of America, Sarasota, Fl, US) s.c. Two-hundred μL of 5 mg/mL dimethylbenzantracene (DMBA, Sigma Aldrich, St. Louis, MO, US) dissolved in corn oil was administered by oral gavage once per week for 7 weeks.

### Immune Checkpoint Blockade

Double or single immune checkpoint blockade was applied by repeated intraperitoneal injections of monoclonal antibody specific to PD-1 (200 μg, Clone 29F.1A12, BioXcell, West Lebanon, NH, USA) or CTLA-4 (200 μg, Clone 9D9, BioXcell) at day 6, 9 and 12 upon initiation of the treatment with lurbinectedin. Animals were monitored regularly and the tumour growth was documented until ethical end-points were reached. Statistical analysis was performed employing 2-way ANOVA analysis followed by Bonferroni's test comparing to control conditions (*p<0.05, **p<0.01 and *p<0.001).

### Statistical Procedures

Unless otherwise specified, experiments were performed in quadruplicate instances. Data were analyzed with the freely available software R (https://www.r-project.org). Significances were calculated using a student t-test with Welch correction. Thresholds for each assay were applied based on the Gaussian distribution of positive and negative controls. In vivo tumour growth was analyzed with the help of the TumGrowth software package (26) freely available at https://github.com/kroemerlab.

### REFERENCES

1. Casares N, Pequignot M O, Tesniere A, et al. Caspase-dependent immunogenicity of doxorubicin-induced tumor cell death. J Exp Med. 2005; 202 (12): 1-9. doi: 10.1084/jem.20050915.
2. Mattarollo S R, Loi S, Duret H, Ma Y, Zitvogel L, Smyth M J. Pivotal role of innate and adaptive immunity in anthracycline chemotherapy of established tumors. Cancer Res. 2011; 71 (14): 4809-4820. doi: 10.1158/0008-5472.CAN-11-0753.
3. Obeid M, Tesniere A, Ghiringhelli F, et al. Calreticulin exposure dictates the immunogenicity of cancer cell death. Nat Med. 2007; 13 (1): 54-61. doi: 10.1038/nm1523.
4. Ray-Coquard I, Cropet C, Van Glabbeke M, et al. Lymphopenia as a prognostic factor for overall survival in advanced carcinomas, sarcomas, and lymphomas. Cancer Res. 2009; 69 (13): 5383-5391. doi: 10.1158/0008-5472.CAN-08-3845.
5. Fridman W H, Zitvogel L, Sautes-Fridman C, Kroemer G. The immune contexture in cancer prognosis and treatment. Nat Rev Clin Oncol. 2017; 14 (12): 717-734. doi: 10.1038/nrclinonc.2017.101.
6. Lesterhuis W J, Haanen J B, Punt C J. Cancer immunotherapy-revisited. Nat Rev Drug Discovery. 2011; 10 (8): 591-600. doi: 10.1038/nrd3500.
7. Zitvogel L, Kepp O, Kroemer G. Immune parameters affecting the efficacy of chemotherapeutic regimens. Nat Rev Clin Oncol. 2011; 8 (3): 151-160. doi: 10.1038/nrclinonc.2010.223.
8. Zappasodi R, Pupa S M, Ghedini G C, et al. Improved clinical outcome in indolent B-cell lymphoma patients vaccinated with autologous tumor cells experiencing immunogenic death. Cancer Res. 2010; 70 (22): 9062-9072. doi: 10.1158/0008-5472.CAN-10-1825.
9. Fucikova J, Kralikova P, Fialova A, et al. Human tumor cells killed by anthracyclines induce a tumor-specific immune response. Cancer Res. 2011; 71 (14): 4821-4833. doi: 10.1158/0008-5472.CAN-11-0950.
10. Ghiringhelli F, Apetoh L, Tesniere A, et al. Activation of the NLRP3 inflammasome in dendritic cells induces IL-1beta-dependent adaptive immunity against tumors. Nat Med. 2009; 15 (10): 1170-1178. doi: 10.1038/nm.2028.
11. Michaud M, Martins I, Sukkurwala A Q, et al. Autophagy-dependent anticancer immune responses induced by chemotherapeutic agents in mice. Science. 2011; 334 (6062): 1573-1577. doi: 10.1126/science.1208347.
12. Apetoh L, Ghiringhelli F, Tesniere A, et al. The interaction between HMGB1 and TLR4 dictates the outcome of anticancer chemotherapy and radiotherapy. Immunol Rev. 2007; 220:47-59. doi: 10.1111/j.1600-065X.2007.00573.x.
13. Tesniere A, Schlemmer F, Boige V, et al. Immunogenic death of colon cancer cells treated with oxaliplatin. Oncogene. 2010; 29 (4): 482-491. doi: 10.1038/onc.2009.356.
14. Sistigu A, Yamazaki T, Vacchelli E, et al. Cancer cell-autonomous contribution of type I interferon signaling to the efficacy of chemotherapy. Nat Med. 2014; 20 (11): 1301-1309. doi: 10.1038/nm.3708.
15. Apetoh L, Ghiringhelli F, Tesniere A, et al. Toll-like receptor 4-dependent contribution of the immune system to anticancer chemotherapy and radiotherapy. Nat Med. 2007; 13 (9): 1050-1059. doi: 10.1038/nm1622.
16. Ma Y, Adjemian S, Mattarollo S R, et al. Anticancer chemotherapy-induced intratumoral recruitment and differentiation of antigen-presenting cells. Immunity. 2013; 38 (4): 729-741. doi: 10.1016/j.immuni.2013.03.003.
17. Vacchelli E, Ma Y, Baracco E E, et al. Chemotherapy-induced antitumor immunity requires formyl peptide receptor 1. Science. 2015; 350 (6263): 972-978. doi: 10.1126/science.aad0779.
18. Kroemer G, Senovilla L, Galluzzi L, Andre F, Zitvogel L. Natural and therapy-induced immunosurveillance in breast cancer. Nat Med. 2015; 21 (10): 1128-1138. doi: 10.1038/nm.3944.
19. Vacchelli E, Enot D P, Pietrocola F, Zitvogel L, Kroemer G. Impact of pattern recognition receptors on the prognosis of breast cancer patients undergoing adjuvant chemotherapy. Cancer Res. 2016; 76 (11): 3122-3126. doi: 10.1158/0008-5472.CAN-16-0294.
20. Santamaría Nuñez G, Robles C M, Giraudon C, et al. Lurbinectedin specifically triggers the degradation of phosphorylated RNA polymerase II and the formation of DNA breaks in cancer cells. Mol Cancer Ther. 2016; 15 (10): 2399-2412. doi: 10.1158/1535-7163.MCT-16-0172.
21. Peltonen K, Colis L, Liu H, et al. A targeting modality for destruction of RNA polymerase I that possesses anticancer activity. Cancer Cell. 2014; 25 (1): 77-90. doi: 10.1016/j.ccr.2013.12.009.
22. Pietrocola F, Pol J, Vacchelli E, et al. Caloric restriction mimetics enhance anticancer immunosurveillance. Cancer Cell. 2016; 30 (1): 147-160. doi: 10.1016/j.ccell.2016.05.016.
23. Martins I, Kepp O, Schlemmer F, et al. Restoration of the immunogenicity of cisplatin-induced cancer cell death by endoplasmic reticulum stress. Oncogene. 2011; 30 (10): 1147-1158. doi: 10.1038/onc.2010.500.
24. Iribarren K, Buque A, Mondragon L, et al. Anticancer effects of anti-CD47 immunotherapy in vivo. Oncoimmunology. 2019; 8 (3): 1550619. doi: 10.1080/2162402X.2018.1550619.
25. Zhou H, Mondragon L, Xie W, et al. Oncolysis with DTT-205 and DTT-304 generates immunological memory in cured animals. Cell Death Dis. 2018; 9 (11): 1086. doi: 10.1038/s41419-018-1127-3.
26. Enot D P, Vacchelli E, J acquelot N, Zitvogel L, TumGrowth: K G. An open-access web tool for the statistical analysis of tumor growth curves. Oncoimmunology. 2018; 7 (9): e1462431. doi: 10.1080/2162402X.2018.1490854.

1. A method of treatment of lung cancer, the method comprising administering lurbinectedin to a patient in need thereof, wherein lurbinectedin is administered in combination with atezolizumab; and wherein lurbinectedin is administered every 21 days by intravenous infusion at a dose of 3.2 mg/m².

2. The method according to claim 1, wherein atezolizumab is administered every 21 days.

3. The method according to claim 1, wherein lurbinectedin is administered as a 1-hour infusion.

4. The method according to claim 1, wherein lurbinectedin and atezolizumab are administered concurrently, separately or sequentially.

5. The method according to claim 1, wherein lurbinectedin is administered initially, followed by atezolizumab.

6. The method according to claim 1, wherein multiple administrations of either lurbinectedin, or atezolizumab, or both, are given.

7. The method according to claim 1, wherein atezolizumab is administered by intravenous infusion.

8. The method according to claim 1, wherein atezolizumab is administered subcutaneously.

9. The method according to claim 1, wherein the lung cancer expresses PD-L1.

10. The method according to claim 1, wherein the method further comprises determining whether the tumor to be treated expresses PD-L1 prior to beginning treatment.

11. The method according to claim 1, wherein the treatment results in one or more of: reduction in tumor size; delay in growth of tumor; prolongation of life of the patient; delay in disease progression; remission.

12. The method according to claim 1, wherein the method comprises prolonging survival of a patient having a lung cancer, delaying disease progression of a lung cancer in a patient and/or reducing or delaying growth of a lung cancer tumor.

13. The method according to claim 2, wherein lurbinectedin is administered as a 1-hour infusion.

14. The method according to claim 13, wherein lurbinectedin and atezolizumab are administered concurrently, separately or sequentially.

15. The method according to claim 14, wherein atezolizumab is administered by intravenous infusion.

16. The method according to claim 15, wherein the lung cancer expresses PD-L1.

17. The method according to claim 16, wherein the method further comprises determining whether the tumor to be treated expresses PD-L1 prior to beginning treatment.

18. The method according to claim 17, wherein the treatment results in one or more of: reduction in tumor size; delay in growth of tumor; prolongation of life of the patient; delay in disease progression; remission; and/or
wherein the method comprises prolonging survival of a patient having a lung cancer, delaying disease progression of a lung cancer in a patient and/or reducing or delaying growth of a lung cancer tumor.

19. The method according to claim 14, wherein atezolizumab is administered subcutaneously.

20. The method according to claim 19, wherein the lung cancer expresses PD-L1.

21. The method according to claim 20, wherein the method further comprises determining whether the tumor to be treated expresses PD-L1 prior to beginning treatment.

22. The method according to claim 21, wherein the treatment results in one or more of: reduction in tumor size; delay in growth of tumor; prolongation of life of the patient; delay in disease progression; remission; and/or
wherein the method comprises prolonging survival of a patient having a lung cancer, delaying disease progression of a lung cancer in a patient and/or reducing or delaying growth of a lung cancer tumor.

23. The method according to claim 3, wherein lurbinectedin and atezolizumab are administered concurrently, separately or sequentially.

24. The method according to claim 23, wherein atezolizumab is administered by intravenous infusion.

25. The method according to claim 24, wherein the lung cancer expresses PD-L1.

26. The method according to claim 25, wherein the method further comprises determining whether the tumor to be treated expresses PD-L1 prior to beginning treatment.

27. The method according to claim 23, wherein atezolizumab is administered subcutaneously.

28. The method according to claim 27, wherein the lung cancer expresses PD-L1.

29. The method according to claim 28, wherein the method further comprises determining whether the tumor to be treated expresses PD-L1 prior to beginning treatment.

30. The method according to claim 29, wherein the treatment results in one or more of: reduction in tumor size; delay in growth of tumor; prolongation of life of the patient; delay in disease progression; remission; and/or
wherein the method comprises prolonging survival of a patient having a lung cancer, delaying disease progression of a lung cancer in a patient and/or reducing or delaying growth of a lung cancer tumor.
